(19) United States
(12) Patent Application Publication
(10) Pub. No.: US 2018/0221425 A1
(43) Pub. Date: Aug. 9, 2018

(54) COMPOSITIONS AND METHODS FOR TREATING WOUNDS

(71) Applicant: RED SWAN LTD., Tel-Aviv (IL)

(72) Inventors: ORLY BEN-LULU, Tel-Aviv (IL); EFRAT ALGRESSI, Tel-Aviv (IL)

(73) Assignee: Red Swan Ltd., Tel-Aviv (IL)

(21) Appl. No.: 15/750,675

(22) PCT Filed: Aug. 9, 2016
(86) PCT No.: PCT/IB2016/054785

§ 371 Date: Feb. 6, 2018

Related U.S. Application Data
(60) Provisional application No. 62/202,843, filed on Aug. 9, 2015.

(51) Int. Cl. A61K 36/28; A61P 43/00; A61K 9/00; A61K 47/44; A61K 47/10; A61K 36/61; A61K 35/644; A61K 9/06; A61K 36/30; A61K 36/185; A61K 36/53; A61K 36/752; A61K 36/537; A61K 36/54; A61K 31/07; A61K 31/355; A61K 36/71; A61K 36/23; A61K 36/738; A61K 36/899; A61K 36/67; A61K 36/85; A61K 45/06

(52) U.S. Cl. CPC A61K 36/28; A61K 45/06; A61K 9/0014; A61K 47/44; A61K 47/10; A61K 36/61; A61K 35/644; A61K 9/06; A61K 36/30; A61K 36/185; A61K 36/53; A61K 36/752; A61K 36/537; A61K 36/54; A61K 31/07; A61K 31/355; A61K 36/71; A61K 36/23; A61K 36/738; A61K 36/899; A61K 36/67; A61K 36/85; A61P 43/00

(57) ABSTRACT

Methods and compositions for treating wounds are described. The compositions include core active ingredients derived from the genera of: Calendula, Achillea Millefolium, Syzygium Aromaticum and Helichrysum. The methods include mixing the core active ingredients, preferably with other secondary active ingredients, adjuvants or excipients, to form the composition and administering the composition by topically applying an effective amount of the composition onto a wound or incision of a patient.

### TECHNICAL FIELD

The present invention generally relates to the treatment of superficial wounds, lesions and sores. More particularly, the present invention pertains to compositions and methods for treating, ameliorating, rehabilitating and/or expediting recuperation from superficial and deep wounds, lesions, sores as well as effectively reducing the amount of scar tissue resulting recuperation, thus contributing to an enhanced visual aesthetic effect of the rehabilitated tissue.

### BACKGROUND ART

Wound healing is an intricate process where the skin or other body tissue repairs itself after injury. The healing process is divided into predictable phases: blood clotting (hemostasis), inflammation, the growth of new tissue (proliferation), and the remodeling of tissue (maturation). In normal skin, the epidermis (surface layer) and dermis (deeper layer) form a protective barrier against the external environment. When the barrier is broken, an orchestrated cascade of biochemical events is quickly set into motion to repair the damage.

Inflammation plays roles in fighting infection, clearing debris and inducing the phase. It is a necessary part of healing but can lead to tissue damage if it lasts too long. Reducing inflammation is frequently a goal in therapeutic settings and lasts as long as there is debris in the wound. A wound compromises the immune system. If, the system is unable to clear the debris from the wound, dead tissue, or microbial biofilm from the wound, then these factors may prolong the inflammatory phase and prevent the wound from properly complete the proliferation phase of healing. This in turn can lead to a chronic wound. Injuries are mostly treated with antibiotic formulations to prevent infection, but carry negative side-effects such as breeding bacterial resistance and causing contact dermatitis. Herbal or natural ingredient based ointments are used to spread over open wounds to diminish such side-effects. Such ointments contain mostly particular vitamins such as A and E, minerals such as zinc and extracts of plants known for their therapeutic or soothing effect such as Aloe Vera. Both antibiotic formulations and currently available natural ointments do not provide a comprehensive solution for different types of wounds, superficial or deep, unintentional or deliberate for example cuts made during surgery. Further, scarring which is part of the healing process of a wound is almost always inevitable at the wound site. To partially prevent that different stitching methods and compositions are used in the art.

### SUMMARY OF THE INVENTION

The present invention addresses the need for a universal composition for treatment of different types of wounds.

It is an object of the present invention to provide a composition that is based on natural herb-based ingredients.

It is another object of the present invention to provide a composition for treating wounds at different stages of healing.

It is yet another object of the present invention to provide a composition for treating wounds, of iatrogenic trauma.

In one aspect, the present invention provides natural substantially herb-based compositions for for treating, ameliorating, rehabilitating and/or expediting recuperation from superficial and deep wounds, lesions and/or sores.

Particularly, the compositions of the present invention may be used to treat surgically or accidentally induced wounds by effectively reducing the amount of scar tissue resulting recuperation, thereby contributing to an enhanced visual aesthetic effect of the rehabilitated tissue.

Furthermore, the compositions of the present invention are suitable for treating different types of wounds selected from burns, scrapes, shallow and deep cuts, rashes, cuts formed by surgical procedures, athlete's foot, blisters, diabetic ulcers, pressure ulcers, surgical incisions, wounds caused by any type of herpes simplex virus 1 and 2 (HSV-1, HSV-2) including Herpetic gingivostomatitis, Herpes labialis, Herpes, Herpes genitalis, Herpetic Whitlow, Herpes gladiatorum, Herpesviral Encephalitis, Herpesviral Meningitis and Herpes Esophagitis, skin conditions, e.g. psoriasis and infected ingrown toenails.

In one particular aspect, the present invention provides natural herb-based compositions that comprise active ingredients and supporting, enhancing, activating and/or enabling adjuvant and formulation for the delivery of active ingredients.

Further, in still another particular embodiment, the combined effect of these active ingredients expresses in a synergistic effect, where the co-existence and activity of the active ingredients improve the efficacy of the entire mixture in treating, ameliorating, rehabilitating and/or expediting recuperation from superficial and deep wounds, lesions and/or sores.

Furthermore, the compositions of the present invention effectively improve chances of regeneration of healthy tissue without scarring or at least with minimal scarring even in deep cuts and in particular intentionally made cuts made in surgical procedures.

In one particular embodiment, the compositions of the present invention may be applied to a wound at any of the different stages of healing. Specifically, the compositions of the present invention are suitable for application in the blood clotting (hemostasis), inflammation, the growth of new tissue (proliferation), and the remodeling of tissue (maturation) of wound. In any of these phases of wound healing, the compositions of the present invention are suitable for enhancing, supporting, regulating and sustaining rehabilitation or regeneration of tissue in different types of wounds.

### Definitions

The term floral waters as referred to herein is to be construed as herbal distillates, also known as floral waters, hydrosols, hydrolates, herbal waters, and essential waters, are aqueous products of distillation. Herbal distillates are colloidal suspensions of essential oils as well as water-soluble components obtained by steam distillation or hydrodistillation, a variant of steam distillation, from plants/herbs. These herbal distillates have uses as flavorings and cosmetic skin care.

The term wounds as referred to herein is to be construed as in non-limiting manner including: burns, scrapes, shallow and deep cuts, rashes, cuts resulting surgical incisions, athlete's foot, blisters, diabetic ulcers, pressure ulcers, surgical incisions, wounds caused by any type of herpes simplex virus HSV-1 and/or HSV-2 including Herpetic gingivostomatitis, Herpes labialis, Herpes, Herpes genitalis, Herpetic Whitlow, Herpes gladiatorum, Herpesviral Encephalitis, Herpesviral Meningitis and Herpes Esophagitis, skin disorders, psoriasis and infected ingrown toenails, skin lesions, sores and any scar tissue resulting therefrom.

### DESCRIPTION OF THE DRAWINGS

The present invention will be understood and appreciated more comprehensively from the following detailed description taken in conjunction with the appended drawings in which:

FIG. 1A is a photograph of a freshly sutured incision in the chest area;

FIG. 1B is a photograph of a freshly sutured incision in the groin area;

FIG. 2 is a photograph of the incision in the groin area covered by a composition, in accordance with some embodiments of the present invention;

FIG. 3A is a photograph of the incision in the chest area, after a month;

FIG. 3B is a photograph of the incision in the groin area, after a month;

FIG. 4A is a photograph of the incision in the chest area, after several months;

FIG. 4B is a photograph of the incision in the groin area, after several months;

FIG. 5A is a photograph of the incisions in chest and groin areas, after a year;

FIG. 5B is an enlarged view of the incision in the groin area, after a year;

FIG. 5C is an enlarged view of the incision in the groin area, after a year;

FIG. 6A is a photograph of an infected incision on the middle finger, for which the conventional treatment has failed;

FIG. 6B is yet another photograph of the incision, shown in FIG. 6A;

FIG. 7A is a photograph of the scar, taken after a year, of the incision on the middle finger treated by a composition of an embodiment of the present invention;

FIG. 7B is yet another photograph of the middle finger scar, shown in FIG. 7A.

### DETAILED DISCLOSURE OF EMBODIMENTS

Illustrative embodiments of the invention are described below. In the interest of clarity, not all features of actual implementation are described in this specification. It will of course be appreciated that in the development of any such actual embodiment, numerous implementation-specific decisions must be made to achieve the developers' specific goals, such as compliance with technology- or business-related constraints, which may vary from one implementation to another. Moreover, it will be appreciated that the effort of such a development might be complex and time-consuming, but would nevertheless be a routine undertaking for those of ordinary skill in the art having the benefit of this disclosure.

In accordance with some embodiments of the present invention, the compositions for treating, ameliorating, rehabilitating and/or expediting recuperation from superficial and deep wounds, lesions, sores and effectively reducing the amount of scar tissue resulting recuperation are natural and essentially herb-based, comprising of plant products and/or extracts selected from particular families. In particular, the compositions in accordance with some embodiments of the present invention comprise plants, plant products and/or extracts from at least the following plant genera: Asteroideae-Calenduleae, Asteroideae-Achillea, Myrtaceae-Syzygium as well as Asteraceae-Helichrysum. Moreover, the compositions in accordance with some embodiments of the present invention preferably comprise at least one bee product, namely a honey extract, honey concentrate, raw honey and/or propolis.

In one embodiment the composition of the present invention preferably comprises products and/or extracts from at least the following plant families/genera: Compositae/asteraceae, Boraginaceae, Iridaceaea, Myrtaceae, Lamiaceae, Citrus, Geraniales and Laurales.

In still another embodiment, the compositions of the present invention further comprise plants, plant products and/or extracts selected from the following plant families/genera: Cannabaceae, Ranunculaceae, Bignoniaceae, Lamiaceae, Cupressaceae and Piperaceae.

In still another embodiment, the compositions of the present invention further comprise plants, plant products and/or extracts selected from the following plant families/genera: Apiaceae/Umbelliferae, Geraniales, Lamiaceae, Rosaceae, Gramineae/Poaceae and Verbenaceae.

In still another embodiment, the compositions of the present invention further comprise ingredients selected from Vitamin A and Vitamin E.

Compositions comprising part or all of the plants listed above may be provided in different forms of administration and further in more than one dosage form that complete the pharmaceutical effect of each other and are suitable for co-administration. Accordingly, different forms of administration comprising part or all of the plants listed above and/or plants selected from the plant families listed above may be selected from cream, ointment, emulsion, spray, liquid formulation (drops), natural medium based solution (e.g., honey, oil), patch, gauze, pad or plaster soaked with ointment of the composition and lozenge. Accordingly, different methods may be used to process the plants and produce initial concentrates to prepare the compositions of the present invention. Examples of such concentrates may be extracts, infusions, tinctures, master solutions, hydrosols, decoctions and any other form of plant produce from which the dosage form can be further prepared. Any such form of administration may comprise a selected combination of plants and/or plants of the same plant families listed above, which prove to convey the same or similar pharmaceutical effect as that of the plants listed above.

In view of the above, the present invention also pertains to a kit comprising one or more dosage forms comprising part or all of the plants listed above or plants of the same plant families that prove to convey the same or similar pharmaceutical effect, where the compositions of such kit are suitable for treating, ameliorating or rehabilitating different types of wounds. Further, these one or more dosage forms in the kit are suitable for enhancing, supporting, regulating and sustaining rehabilitation or regeneration of tissue in different types of wounds.

Further, the present invention also pertains to a kit comprising one or more dosage forms comprising part or all of the plants listed above or plants of the same plant families that prove to convey the same or similar pharmaceutical effect, where the compositions of such kit are suitable for application throughout any of the different stages of wound healing. Specifically, the compositions of the present invention are suitable for application in the blood clotting (hemostasis), inflammation, the growth of new tissue (proliferation), and the remodeling of tissue (maturation) stags of wound healing.

In other embodiments of the present invention, the kit comprises one to any number of dosage forms, where each dosage form comprises any one of possible compositions that comprises plants of the plant families listed above. Particularly, such compositions may be selected from any one of examples detailed below.

In still other embodiments of the present invention, the kit comprises one to any number of dosage forms, where each dosage form comprises a combination of the active ingredients listed above and adjuvants and/or excipient. Such adjuvants and/or excipients are provided inter alia by any one of the plants selected from the list of plants or plant families listed above.

Such compositions are also intended to provide efficient, less harmful, absent of side- or long term effects substitute for currently synthetic antibiotics as well as general purpose natural based compositions, such as Aloe Vera cream. Positive effects of the compositions of the present invention are also expressed in post-healing of a wound, where the healing process has been incomplete or resulted in surface or deep scarring of the wounded tissue.

In another aspect, the present invention provides a method for treating different types of wounds. Particularly, the method comprises treatment protocol and administration regimen with one or more compositions comprising plants selected from part or all of the plants and/or plant families listed above. In one particular embodiment, the treatment protocol and administration regimen comprise: (a) diagnosing type of wound in a patient; (b) diagnosing current state of the wound; (c) determining severity of the wound; (d) prescribing appropriate combination of plants of the present invention for treating the wound; (e) selecting appropriate dosage form of administration for treating the wound; (f) applying the dosage form to the wound; (g) reiterating step (f) for a prescribed period of time; (h) reiterating steps (a)-(c); and if required reiterating steps (d)-(g).

The treatment protocol detailed above may be implemented in different ways and until complete or at least satisfactory wound healing is obtained. For example, the administration regimen may be determined to three times a day until full regeneration of skin tissue in the area of the wound. In another example, the composition may be applied instantly in emergency cases such as fire burn, cut of a knife, injury caused after falling or car accident or immediately after surgery incision to prevent infection and stabilize conditions for future recovery.

In one particular example, the compositions of the present invention are suitable for treating different types of wounds selected from burns, scrapes, shallow and deep cuts, rashes, surgical procedures, athlete's foot, blisters, diabetic ulcers, pressure ulcers, cuts made by surgical incisions, wounds caused by any type of herpes simplex virus 1 and 2 (HSV-1, HSV-2) including Herpetic gingivostomatitis, Herpes labialis, Herpes, Herpes genitalis, Herpetic Whitlow, Herpes gladiatorum, Herpesviral Encephalitis, Herpesviral Meningitis and Herpes Esophagitis, skin conditions, e.g. psoriasis and infected ingrown toenails.

The compositions and treatment protocol of the present invention are preferably suitable for self-treatment and self-application by the injured person, particularly when first aid is essential and no medical help is available.

The application of the compositions of the present invention to wounds may be carried out in different ways and provided in different dosage forms, some of which are listed above. Particularly, the compositions of the present invention may be provided in the form of ointment, particularly intended for cuts, burns and resurfacing of scar tissue. For cuts and burns, apply a thick layer on the wound surface as close as possible to the time of its formation; then wrap with pad or gauze with the layer still on the wound. Replace pad and gauze at least three times a day. After healing, i.e., the wound area should no longer be touch-sensitive, remove the pads and gauzes and apply ointment at least three times a day; allow the ointment to be absorbed in the skin until scar formed fades or vanishes.

The spray is more practical for cuts and burns, and may be applied in the following situations: inaccessible wound area—either because it cannot be reached or due to much pain caused upon contact. Application of ointment is less possible in such cases. Accordingly, spray the wounded area from a distance until injured person feels relieved. Then apply ointment. Otherwise, continue with spraying the wounded area. Apply spray, in cases when sterility cannot be kept, for example unclean hands for application of ointment in a non-sterile environment.

A patch optionally applied to a wounded area when mobility of the injured person is required and wound dressing cannot be comfortably done.

In one particular embodiment, the compositions of the present invention are suitable for treating wounds in every phase of body response to formation of wound as follows: severe burns and deep cuts, particularly surgical incisions where the treatment is provided instantly after surgical operation. The impact of treatment with the compositions of the present invention on the wound comprises the following effects: stopping hemorrhaging, preventing and dissolving hematoma, treating infections and secretion of fluids caused by infection, antiseptic effect, fusing resurfacing and remodeling skin tissue, soothing and relieving pain.

### EXAMPLES

### Example 1

Exemplary compositions of the invention is prepared by mixing the following core active ingredients: (1) Calendula infused in olive oil, (2) Achillea Millefolium tincture in alcohol, (3) Syzygium Aromaticum floral water extract, and (4) Helichrysum essential oil, as well as (5) raw honeybee honey. In different variants of the composition, the relative weight amount of Calendula infused in oil to the total weight of the composition has been found to be effective in the range between 10% (w/w) and 20% (w/w). In different variants of the composition, the relative weight amount of Achillea Millefolium tincture to the total weight of the composition has been found to be effective in the range between 4% (w/w) to 25% (w/w). In different variants of the composition, the relative weight amount of Syzygium Aromaticum floral water to the total weight of the composition has been found to be effective in the range between 4% (w/w) to 25% (w/w). In different variants of the composition, the relative weight amount of Helichrysum essential oil to the total weight of the composition has been found to be effective in the range between 1% (w/w) to 10% (w/w). Whereas, the relative weight amount of the raw honeybee honey to the total weight of the composition has been found to be effective in the range between 20% (w/w) to 50% (w/w).

### Example 2

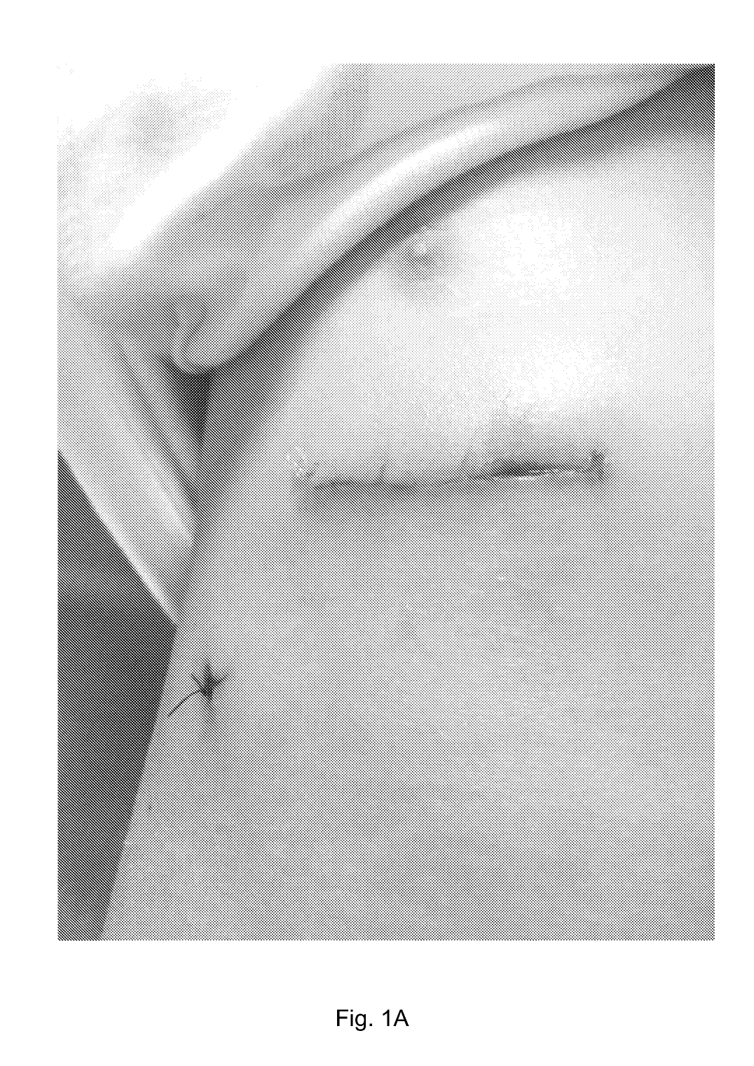

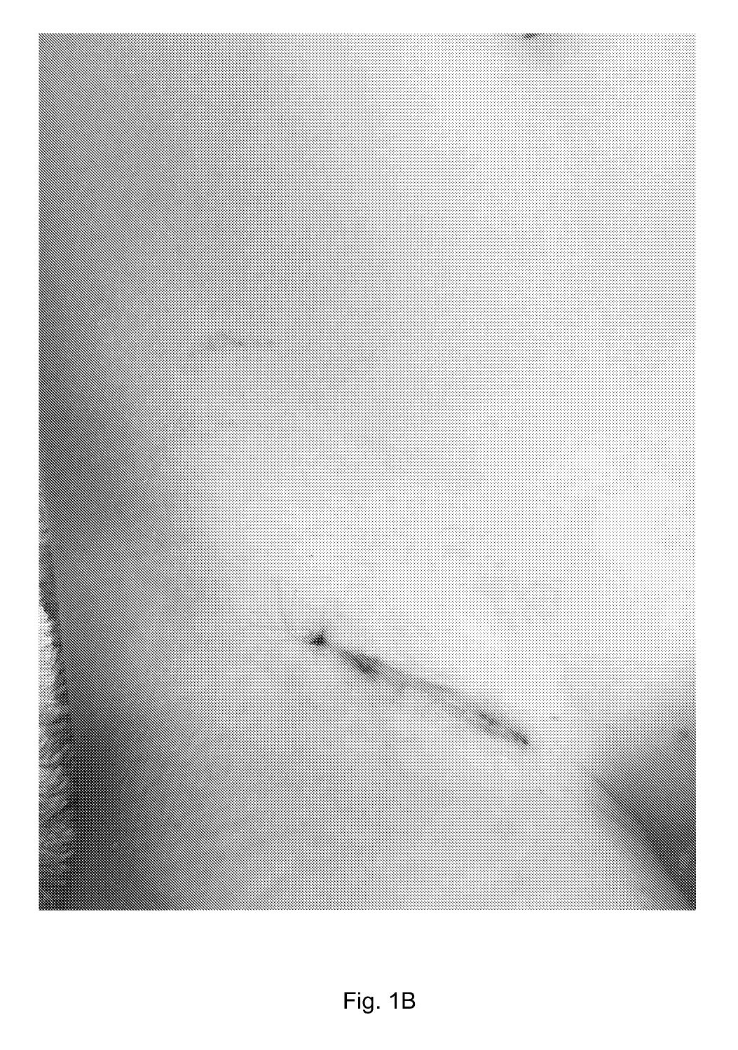

Reference is now made to FIG. 1A to 5C, showing incisions performed on an eight years old female patient, in chest and groin areas. The incision in the chest area hasn't been specially treated and served as a negative control, whereas the incision in the groin area has been intensively treated for a month as well as occasionally throughout the following three to four months, following the incision. The freshly sutured incision in the chest area is shown in FIG. 1A; whereas freshly sutured incision in the groin area is shown in FIG. 1B.

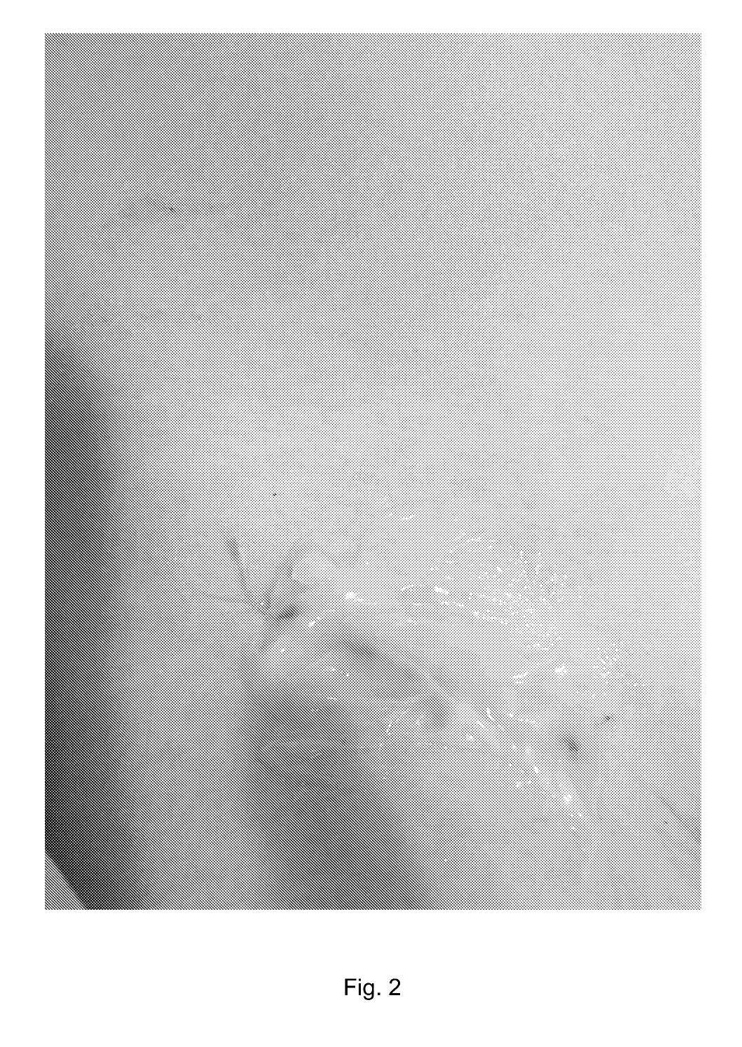

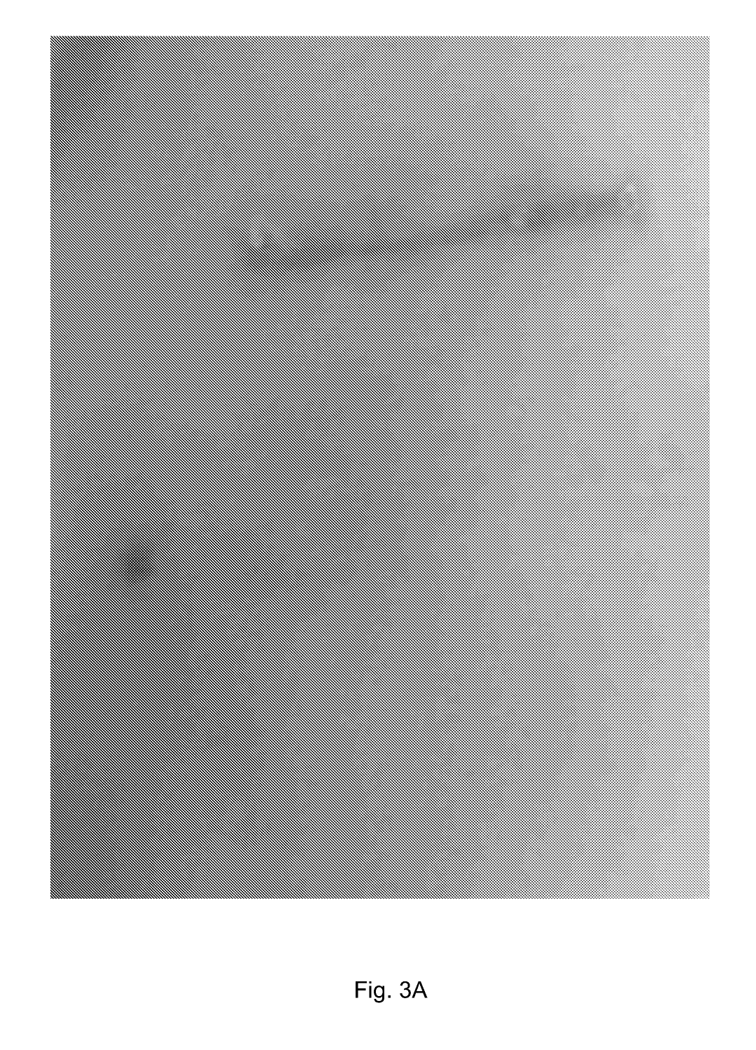

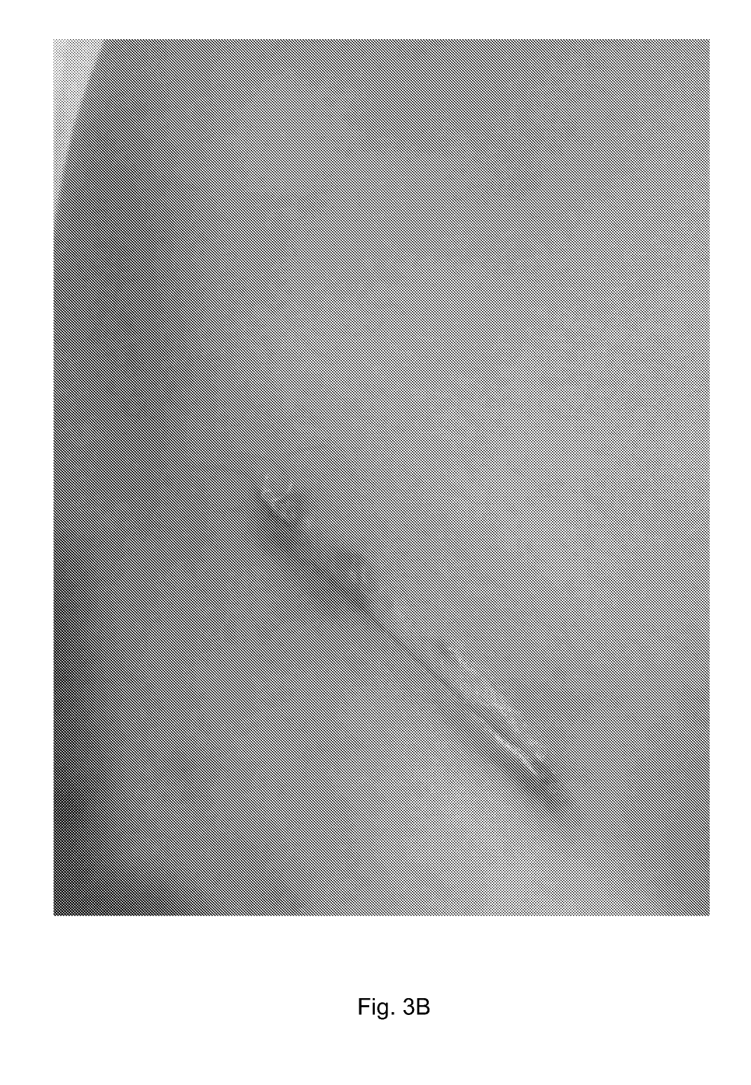

The incision in the groin area has been treated three to four times throughout each day, for a month, by topical application essentially covering the the entire incision, as shown in FIG. 2, by the composition prepared essentially as set forth in Example 1 supra. After approximately a month, both incisions have closed and a wound tissue was formed, as shown in FIG. 3A and FIG. 3B, respectively.

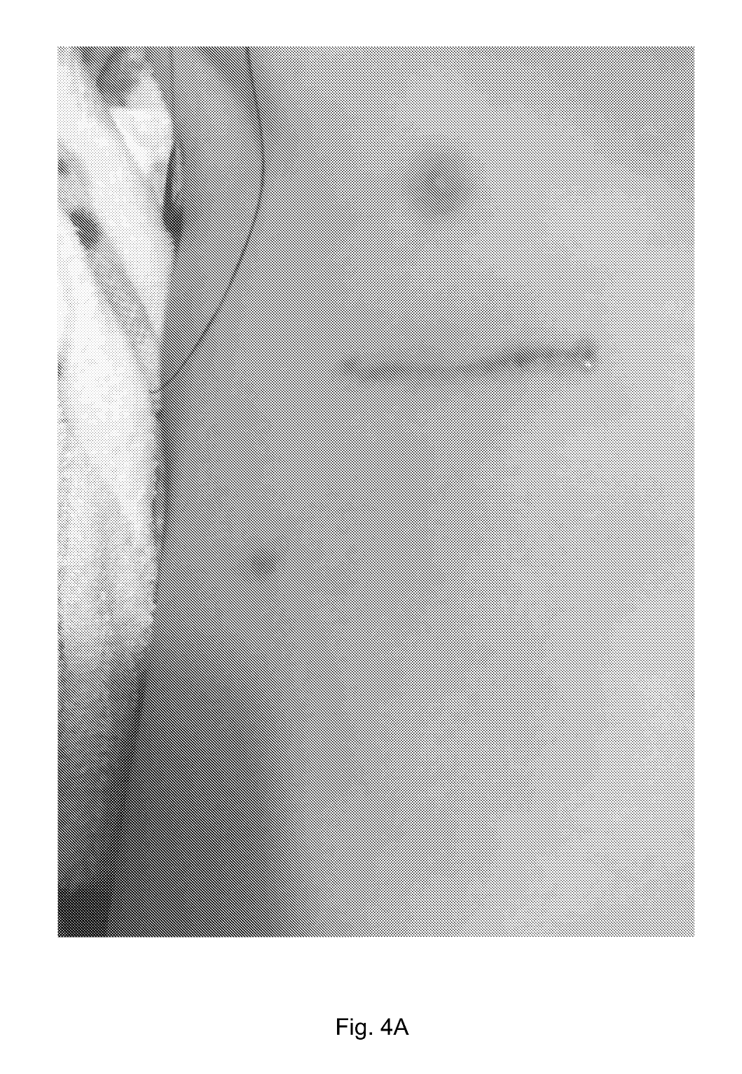

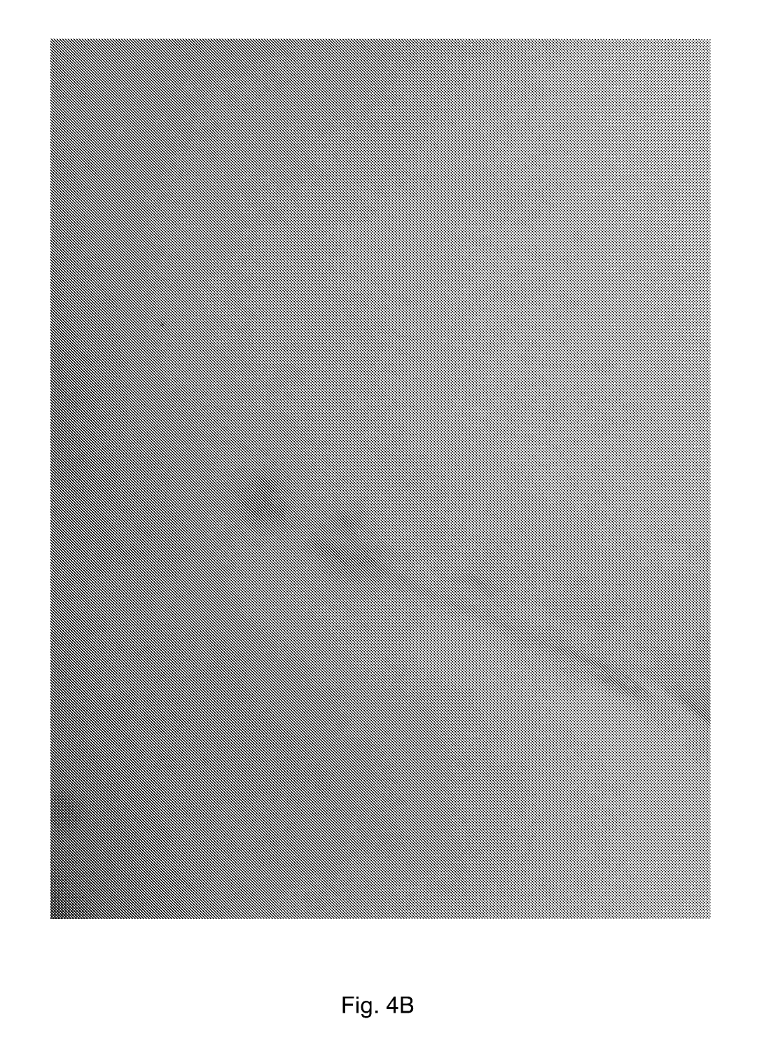

The incision in the groin area has received an occasional topical treatment several timed throughout a week, by a composition of the invention, essentially similarly to the treatment shown in FIG. 2. After the following three to four months, the surgical sutures have fallen out and the primary scar tissue has formed, as shown in FIG. 4A and FIG. 4B, respectively.

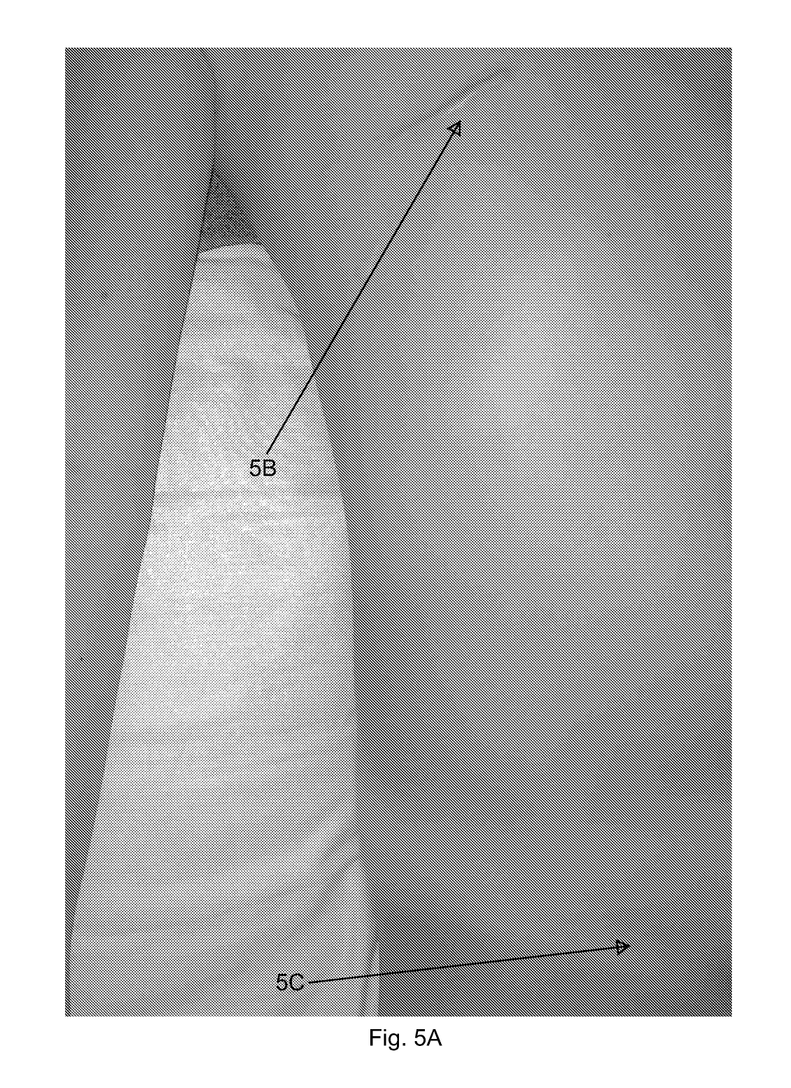

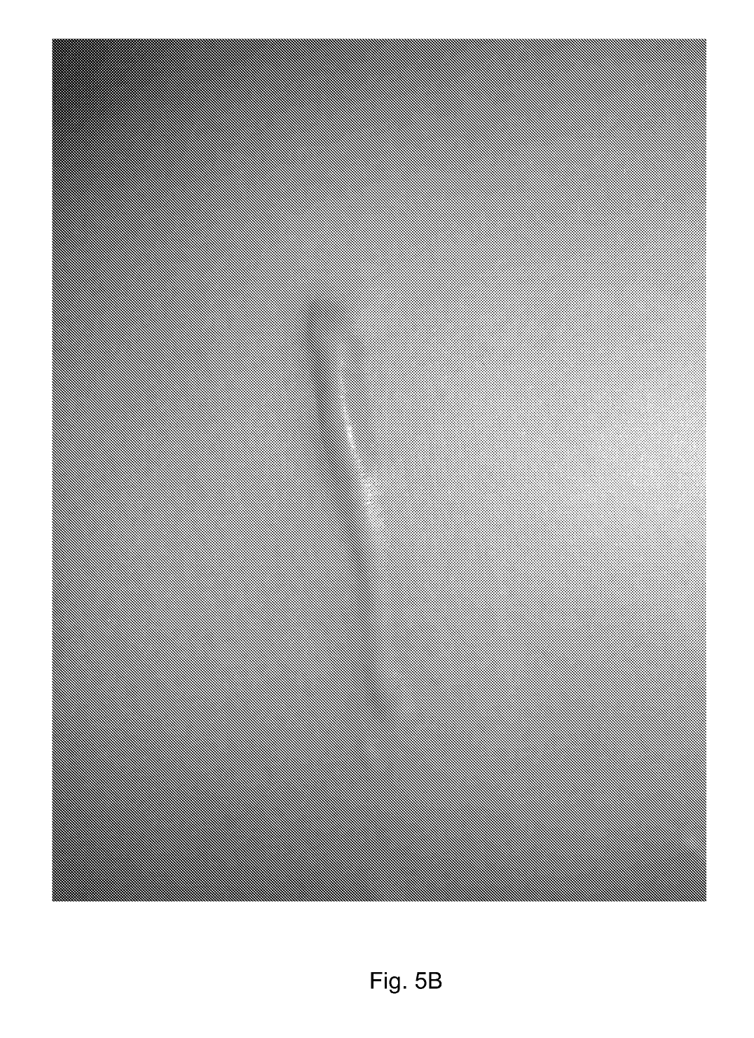

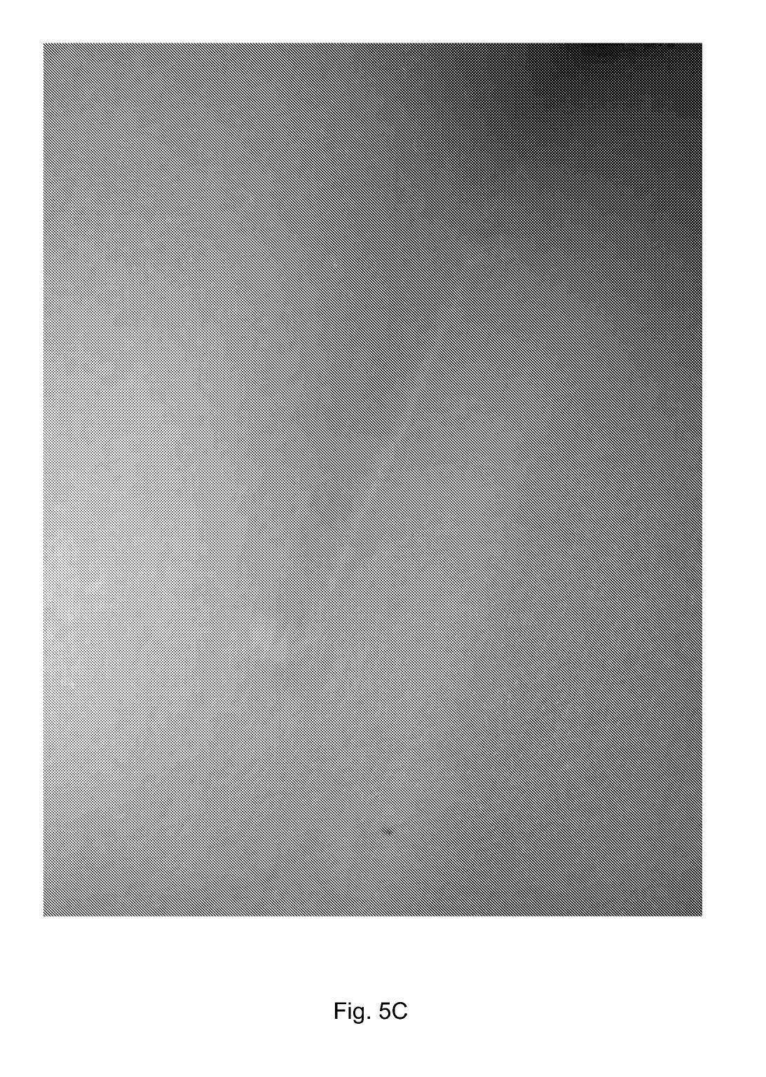

After about a year both incisions have completely healed and a final scar tissue has formed, as shown in FIG. 5A to FIG. 5C. As clearly seen from the comparison between the scar of the incision in the chest area versus the scar of the incision in the groin, the untreated negative control incision in the chest area, shown in FIG. 5B area, has developed a significant, vividly apparent and considerably unaesthetic scar tissue; whereas the incision in the groin area, shown in FIG. 5C, which has been treated with the composition of the present invention, is barely visible and developed a minimal scar tissue, characterized by an apparent and considerably superb aesthetic effect.

### Example 3

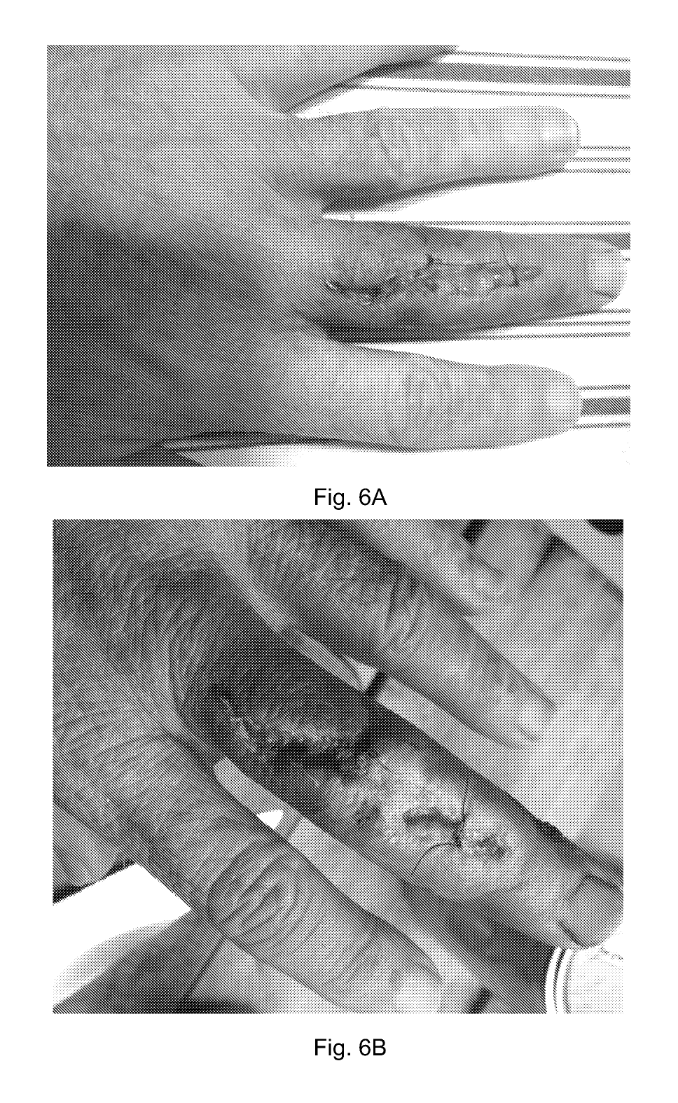

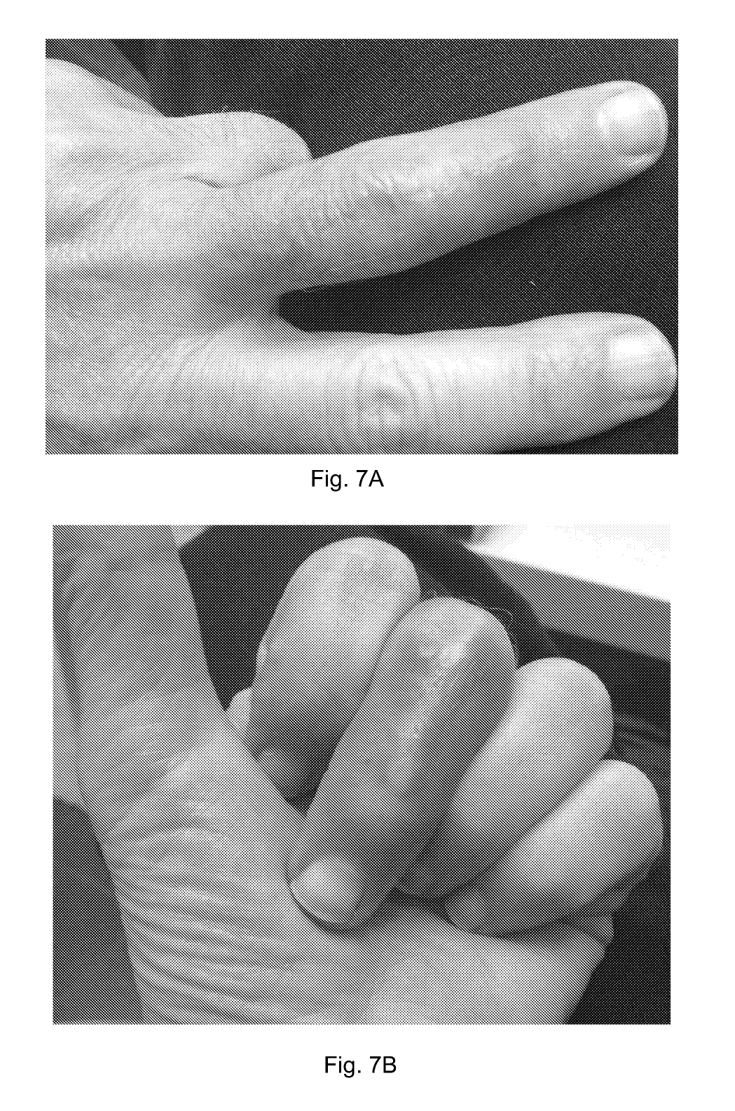

Referring now to FIG. 6A to 7B, where the initial state of a deep severely infected wound inflicted to a middle finger of a male patient, born on 26 Nov. 1956, as well as the final scar tissue of the wound treated by a composition of the invention are shown. The patient has commenced receiving the treatment by a composition of the invention, after the conventional gold-standard medical treatment has failed and the deeply wound and severely infected middle finger of the patient, shown in FIGS. 6A and 6B taken on 25 Dec. 2015, was at a considerably increasing risk of progressing gangrene which would require amputation of the entire middle finger.

Prior to receiving the treatment by a composition of the invention, the patient has undergone surgical suturing as well as iterative clinical debridement of the wound, as a part of the conventional gold-standard medical treatment received in a modern hospital Ichilov Hospital with contemporary equipment in Tel Aviv, Israel. The patient started receiving intensive treatment, including massive topical application of a composition of the invention, by the composition prepared essentially as set forth in Example 1 supra, substantially covering the the entire wound, similarly to the incision shown in FIG. 2, several times throughout each day.

Following merely about two months of intensive treatment by massive topical application of a composition of the invention, the initial deep and severely infected wound on the middle finger of the patient, shown in FIGS. 6A and 6B taken on 25 Dec. 2015, has exhibited a remarkable clinical progress, whilst the wound has essentially completely recuperated and a fresh scar tissue has entirely closed the wound, as shown in FIGS. 7A and 7B taken on 20^{th} of February 2016. The present instance has clearly proven the effectiveness of the composition of the invention not only in achieving a considerable aesthetic effect on the resulting scar tissue but also in accomplishing a considerably beneficial clinical effect, notably where the conventional treatment has essentially failed and the patient was at an increasingly realistic risk of losing the finger.

### Example 4

Another exemplary composition of the invention is prepared by mixing the core active ingredients set forth in Example 1 with at least one of the following secondary active ingredients and/or adjuvants and/or excipients: (1) plant, parts plants, plant products and/or extracts selected from the following plant families/genera: Compositae/Asteraceae, Boraginaceae, Iridaceaea, Myrtaceae, Lamiaceae, Citrus and/or Geraniales, Laurales, as well as (2) at least one of the following ingredients: Vitamin A and Vitamin E.

### Example 5

Yet another exemplary composition of the invention is prepared by mixing the core active ingredients set forth in Example 1 with at least one of the following secondary active ingredients and/or adjuvants and/or excipients: plant, parts plants, plant products and/or extracts selected from the following plant families/genera: Cannabaceae, Ranunculaceae, Bignoniaceae, Lamiaceae, Cupressaceae and Piperaceae.

### Example 6

Still another exemplary composition of the invention is prepared by mixing the core active ingredients set forth in Example 1 with at least one of the following secondary active ingredients and/or adjuvants and/or excipients: plant, parts plants, plant products and/or extracts selected from the following plant families/genera: Apiaceae/Umbelliferae, Lamiaceae, Rosaceae, Gramineae/Poaceae and Verbenaceae.

### Example 7

Yet still another exemplary composition of the invention is prepared by mixing the core active ingredients set forth in Example 1 with at least one of the following secondary active ingredients and/or adjuvants and/or excipients: plant, parts plants, plant products and/or extracts selected from the following plant families/genera: Calendula Officinalis infused in any oil, Symphytum Officinalis tincture in alcohol, Iris Versicolor tincture in alcohol, Achillea Millefolium tincture in alcohol, Matricaria Chamomile floral water extract, Eugenia Caryophyllus floral water extract, Lavandula Angustifolia essential oil, Citrus Limonum essential oil, Geranium Maculatum and/or Pelargonium Sidoides essential oil, Salvia Officinalis essential oil, Cinnamomum Camphora tincture in alcohol, Tagetes Minuta and Pogostemon Cabin essential oil, as well as at least one of the following ingredients: Vitamin A and Vitamin E.

### Example 8

Yet another exemplary composition of the invention is prepared by mixing the core active ingredients set forth in Example 4 with at least one of the following secondary active ingredients and/or adjuvants and/or excipients: plant, parts plants, plant products and/or extracts selected from the following plant families/genera: Cannabis Sativa, Hydrastis Canadensis, Tabebuia Impestiginosa, Lavandula Angustifolia, Cupressus Sempervirens, Helichrysum Italicum and Piper Nigrum, Cuminum Cyminum, Geranium Maculatum/Pelargonium Sidoides, Rosa Damascena, Cymopogon Nardus and Verbena Officinalis. Moreover, a composition of the invention is prepared by mixing the core active ingredients set forth in Example 4 with at least one of the following secondary active ingredients and/or adjuvants and/or excipients: plant, parts plants, plant products and/or extracts selected from the following plant families/genera: Hydrastis Canadensis tincture in alcohol and/or Crocus Sativus tincture in alcohol, Handroanthus Impetiginosus tincture in alcohol, Cuminum Cyminum floral water extract, Lavandula Angustifolia floral water extract, Geranium Maculatum and/or Pelargonium Sidoides floral water extract, Salvia Officinalis floral water extract, Cupressus Sempervirens essential oil, Rosa Damascena essential oil, Cymopogon Nardus essential oil, Piper Nigrum essential oil, Verbena Officinalis essential oil.

### Example 9

Still another exemplary composition of the invention is prepared by mixing of the ingredients set forth in Example 4 with at least one of the following secondary active ingredients and/or adjuvants and/or excipients: Lutein, Allantoin, Salicylic acid, Hemp oil or hempseed oil, Azulen, Chamazulene, Eugenol, Terpinen-4-ol, Limonene Camphor, Tagetone and Patchoulol. In addition to the compounds in the current example, composition of the invention is prepared by mixing at least one of the following adjuvants and/or excipients: Nerolidol, Berberine, Dehydro-alpha-apachone, Caffeic acid, Sabinene, Neryl acetate and Beta-Caryophyllene, Cuminaldehyde, Myrcene, Citronellal and Verbascoside.

### Example 10

Still yet another exemplary composition of the invention is prepared by mixing the core active ingredients set forth in Example 1 with at least one of the following secondary active ingredients and/or adjuvants and/or excipients: plant, parts plants, plant products and/or extracts selected from the following plant families/genera: Compositae/asteraceae, Boraginaceae, Iridaceaea, Myrtaceae, Lamiaceae, Citrus, Geraniales, Laurales, Cannabaceae, Ranunculaceae, Bignoniaceae, Lamiaceae, Cupressaceae, Piperaceae, Apiaceae/Umbelliferae, Lamiaceae, Rosaceae, Gramineae/Poaceae and Verbenaceae, and the ingredients Vitamin A and Vitamin E, where the relative weight amount of these compounds varies between 0.1% w/w to 25% w/w.

### Example 11

Still another exemplary composition of the invention is prepared by mixing of the core active ingredients set forth in Example 1 with at least one of the following secondary active ingredients and/or adjuvants and/or excipients: Lutein, Allantoin, Salicylic acid, Azulen, Chamazulene, Eugenol, Terpinen-4-ol, Limonene Camphor, Tagetone and Patchoulol. In addition to the compounds in the current example, composition of the invention is prepared by mixing at least one of the following adjuvants and/or excipients: Nerolidol, Berberine, Dehydro-alpha-apachone, Caffeic acid, Sabinene, Neryl acetate and Beta-Caryophyllene, Cuminaldehyde, Myrcene, Citronellal and Verbascoside, where the relative weight amount of any one of these compounds varies between 0.1% w/w to 10% w/w.

### BIBLIOGRAPHICAL CITATION LIST

### Non Patent Literature

Herbal distillate—In Wikipedia, The Free Encyclopedia. Retrieved Aug. 7, 2016, from https://en.wikipedia.org/w/index.php?title=Herbal_distillate&oldid=732778729

It will be appreciated by persons skilled in the art that the present invention is not limited by what has been particularly shown and described herein above. Rather the scope of the invention is defined by the claims which follow:

1. A method of treating wounds comprises:
(a) providing core active ingredients comprising:
(I) an effective amount of Calendula infused in oil;
(II) an effective amount of Achillea Millefolium tincture in alcohol;
(III) an effective amount of Syzygium Aromaticum floral water extract;
(IV) an effective amount of Helichrysum essential oil;
(V) an effective amount of at least one honeybee product selected from the group consisting of: raw honey, honey extract, honey concentrate and propolis;
(b) mixing said core active ingredients to produce an essentially homogenous composition;
(c) administering said composition to a patient, wherein said administering comprising topically applying an effective amount of said composition onto a wound or incision;
(d) iteratively administering said composition to said patient.

2. The method of treating wounds as set forth in claim 1, further comprises mixing said core active ingredients with a suitable pharmaceutical excipient, to formulate said composition into an ointment.

3. The method of treating wounds as set forth in claim 1, wherein said administering comprises spraying said composition onto said wound or incision.

4. The method of treating wounds as set forth in claim 1, wherein relative weight of said Calendula infused in oil to a total weight of said composition ranges between 10% (w/w) and 20% (w/w); wherein relative weight of said Achillea Millefolium tincture to said total weight of said composition ranges between 4% (w/w) to 25% (w/w); relative weight of said Syzygium Aromaticum floral water to said total weight of said composition ranges between 4% (w/w) to 25% (w/w); relative weight of said Helichrysum essential oil to said total weight of said composition ranges between 1% (w/w) to 10% (w/w), and relative weight of said honeybee product to said total weight of said composition ranges between 20% (w/w) to 50% (w/w).

5. The method of treating wounds as set forth in claim 1, wherein said composition further comprises at least one secondary active ingredient and/or adjuvant selected from the group consisting of: Calendula Officinalis infused in oil, Symphytum Officinalis tincture in alcohol, Iris Versicolor tincture in alcohol, Achillea Millefolium tincture in alcohol, Matricaria Chamomile floral water extract, Eugenia Caryophyllus floral water extract, Lavandula Angustifolia essential oil, Citrus Limonum essential oil, Geranium Maculatum, Pelargonium Sidoides essential oil, Salvia Officinalis essential oil, Cinnamomum Camphora tincture in alcohol, Tagetes Minuta, Pogostemon Cabin, Vitamin A and Vitamin E.

6. The method of treating wounds as set forth in claim 1, wherein said composition further comprises at least one secondary active ingredient and/or adjuvant selected from the group consisting of: Hydrastis Canadensis tincture in alcohol and/or Crocus Sativus tincture in alcohol, Handroanthus Impetiginosus tincture in alcohol, Cuminum Cyminum floral water extract, Lavandula Angustifolia floral water extract, Geranium Maculatum, Pelargonium Sidoides floral water extract, Salvia Officinalis floral water extract, Cupressus Sempervirens essential oil, Rosa Damascena essential oil, Cymopogon Nardus essential oil, Piper Nigrum essential oil and Verbena Officinalis essential oil.

7. The method of treating wounds as set forth in claim 1, wherein said composition further comprises at least one secondary active ingredient and/or adjuvant comprising a plant part, plant product or plant extract of a plant from a family/genus selected from the group consisting of: Compositae/Asteraceae, Boraginaceae, Iridaceaea, Myrtaceae, Lamiaceae, Citrus, Geraniales, Laurales, Cannabaceae, Ranunculaceae, Bignoniaceae, Lamiaceae, Cupressaceae, Piperaceae, Apiaceae/Umbelliferae, Lamiaceae, Rosaceae, Gramineae/Poaceae, Verbenaceae, Symphytum Officinalis, Iris Versicolor, Achillea Millefolium, Matricaria Chamomile, Eugenia Caryophyllus, Lavandula Angustifolia, Citrus Limonum, Geranium Maculatum, Pelargonium Sidoides, Salvia Officinalis, Cinnamomum Camphora, Tagetes Minuta, Pogostemon Cabin, Cannabis Sativa, Hydrastis Canadensis, Tabebuia Impestiginosa, Lavandula Angustifolia, Cupressus Sempervirens, Helichrysum Italicum, Piper Nigrum, Cuminum Cyminum, Geranium Maculatum, Pelargonium Sidoides, Rosa Damascena, Cymopogon Nardus and Verbena Officinalis.

8. The method of treating wounds as set forth in claim 1, wherein said composition further comprises at least one secondary active ingredient and/or adjuvant selected from the group consisting of: Vitamin A, Vitamin E, Lutein, Allantoin, Salicylic acid, Azulen, Chamazulene, Eugenol, Terpinen-4-ol, Limonene Camphor, Tagetone, Patchoulol, Nerolidol, Berberine, Dehydro-alpha-apachone, Caffeic acid, Sabinene, Neryl acetate, Beta-Caryophyllene, Cuminaldehyde, Myrcene, Citronellal, Verbascoside.

9. A composition for treating wounds comprises:
(a) an effective amount of a product or extract of Calendula;
(b) an effective amount of a product or extract of Achillea Millefolium;
(c) an effective amount of a product or extract of Syzygium Aromaticum;
(d) an effective amount of a product or extract of Helichrysum;
(e) an effective amount of at least one honeybee product selected from the group consisting of: raw honey, honey extract, honey concentrate and propolis.

10. The composition for treating wounds as set forth in claim 9, wherein relative weight of said Calendula product to a total weight of said composition ranges between 10% (w/w) and 20% (w/w); wherein relative weight of said Achillea Millefolium product to said total weight of said composition ranges between 4% (w/w) to 25% (w/w); relative weight of said Syzygium Aromaticum product to said total weight of said composition ranges between 4% (w/w) to 25% (w/w); relative weight of said Helichrysum product to said total weight of said composition ranges between 1% (w/w) to 10% (w/w), and relative weight of said honeybee product to said total weight of said composition ranges between 20% (w/w) to 50% (w/w).

11. The composition for treating wounds as set forth in claim 9, wherein further comprises mixing said core active ingredients with a suitable pharmaceutical excipient, to formulate said composition into an ointment.

12. The composition for treating wounds as set forth in claim 9, wherein said administering comprises spraying said composition onto said wound, incision or skin disorder.

13. The composition for treating wounds as set forth in claim 9, wherein said composition further comprises at least one secondary active ingredient and/or adjuvant comprising a plant part, plant product or plant extract of a plant from a family selected from the group consisting of: Compositae/Asteraceae, Boraginaceae, Iridaceaea, Myrtaceae, Lamiaceae, Citrus, Geraniales, Laurales, Cannabaceae, Ranunculaceae, Bignoniaceae, Lamiaceae, Cupressaceae, Piperaceae, Apiaceae/Umbelliferae, Lamiaceae, Rosaceae, Gramineae/Poaceae, Verbenaceae.

14. The composition for treating wounds as set forth in claim 9, wherein said composition further comprises at least one secondary active ingredient and/or adjuvant comprising a plant part, plant product or plant extract of a plant from a genus selected from the group consisting of: Symphytum Officinalis, Iris Versicolor, Achillea Millefolium, Matricaria Chamomile, Eugenia Caryophyllus, Lavandula Angustifolia, Citrus Limonum, Geranium Maculatum, Pelargonium Sidoides, Salvia Officinalis, Cinnamomum Camphora, Tagetes Minuta, Pogostemon Cabin, Cannabis Sativa, Hydrastis Canadensis, Tabebuia Impestiginosa, Lavandula Angustifolia, Cupressus Sempervirens, Helichrysum Italicum, Piper Nigrum, Cuminum Cyminum, Geranium Maculatum, Pelargonium Sidoides, Rosa Damascena, Cymopogon Nardus and Verbena Officinalis.

15. The composition for treating wounds as set forth in claim 9, wherein said composition further comprises at least one secondary active ingredient and/or adjuvant selected from the group consisting of: Vitamin A, Vitamin E, Lutein, Allantoin, Salicylic acid, Azulen, Chamazulene, Eugenol, Terpinen-4-ol, Limonene Camphor, Tagetone, Patchoulol, Nerolidol, Berberine, Dehydro-alpha-apachone, Caffeic acid, Sabinene, Neryl acetate, Beta-Caryophyllene, Cuminaldehyde, Myrcene, Citronellal, Verbascoside.
